(19) United States
(12) Patent Application Publication
(10) Pub. No.: US 2012/0237533 A1
(43) Pub. Date: Sep. 20, 2012

(54) Compositions and Methods for Inducing Apoptosis in Prostate Cancer Cells

(76) Inventors: George Kulik, Winston-Salem, NC (US); Mark E. Welker, Clemmons, NC (US); Freddie R. Salsbury, JR., Winston-Salem, NC (US)

(21) Appl. No.: 13/366,715

(22) Filed: Feb. 6, 2012

Related U.S. Application Data
(63) Continuation-in-part of application No. PCT/US2010/044548, filed on Aug. 5, 2010.
(60) Provisional application No. 61/231,391, filed on Aug. 5, 2009.

(51) Int. Cl. A61K 38/45; A61P 35/00; A61K 39/395

(52) U.S. Cl. 424/183.1; 424/94.5

(57) ABSTRACT

Compositions and methods for inhibiting the growth of cancer cells, particularly prostate cancer cells are disclosed.

This application is a 35 U.S.C. §365(c) application of PCT/US2010/044548 filed Aug. 5, 2010 which in turn claims priority to U.S. Provisional Application No. 61/231,391 filed Aug. 5, 2009, the entire contents of each being incorporated herein by reference.

Pursuant to 35 U.S.C. §202(c) it is acknowledged that the U.S. Government has rights in the invention described, which was made in, part with funds from the National Department of Defense, Grant Number W81XWH-08-0044.

### FIELD OF THE INVENTION

This invention relates to the fields of oncology and modulation of signal transduction pathways for inducing targeted cell death. More specifically, compositions and methods which act synergistically to induce apoptosis in cancer cells, particularly in prostate cancer cells are disclosed which may be used to advantage in innovative treatment modalities for androgen-independent and androgen-dependent prostate carcinomas.

### BACKGROUND OF THE INVENTION

Several publications and patent documents are cited throughout the specification in order to describe the state of the art to which this invention pertains. Each of these citations is incorporated herein by reference as though set forth in full.

The prostate is a walnut-sized gland located between the pubic bone and bladder. As men age, aberrant prostate growth is commonly observed. While benign prostate hyperplasia (BPH) is characterized by urinary tract obstruction due to prostate enlargement, malignant transformation of the prostate is accompanied by uncontrolled growth, invasion, metastasis and ultimately death.

Prostate cancer is the most commonly diagnosed cancer and the second leading cause of cancer death in men annually. Frequently, patients present with local advanced disease and/or detectable distant bone metastasis at initial diagnosis. The only treatment modality recommended for patients with advanced disease is androgen ablation therapy.

Tumor regression and improvement of clinical symptoms have been achieved by several means including castration, the use of diethylstilbestrol or luteinizing hormone releasing hormone agonist (LHRH) to lower circulating serum androgen, and by antagonizing androgen action with antiandrogens, such as, flutamide, cyproterone acetate or CASODEX®. However, the tumor regression observed with these treatment modalities is only temporary. Inevitably, the disease progresses to an androgen-independent state rendering androgen ablation therapy ineffective. Unfortunately, there is no effective therapy available to treat androgen-independent prostate cancers.

Prostate specific antigen (PSA) and prostate specific membrane antigen (PSMA) are sensitive markers for prostate cancer diagnosis and progression. PSA is a serine protease produced by the prostatic ductal epithelial cells which is secreted into the seminal plasma. In normal prostate, a basement membrane acts as a barrier to block PSA from escaping into systemic circulation.

Anti-androgen treatment of prostate cancer causes PSA serum levels in patients to drop precipitously. However, more than 50% of all patients see a steady increase in PSA serum levels six months after surgical castration and/or anti-androgen therapy. This rebound in PSA serum levels indicates that the prostate cancer has become resistant to the anti-androgen treatment. Since PSA expression is controlled mainly by the androgen receptor (AR), it has been suggested that the rebound in PSA serum levels occurs because AR acquires functional activity in the absence of androgen.

Prostate specific membrane antigen (PSMA), a 100 kDa type II membrane glycoprotein with peptidase and folate hydrolase activity, was originally identified as an antigen interacting with the prostate-specific monoclonal antibody, 7E11-05. PSMA serum levels in prostate cancer are significantly higher than those observed in benign prostate hyperplasia or normal prostate, suggesting that enhanced expression of PSMA occurs during prostate cancer progression.

Prostate cancers evolve to become androgen-independent and refractory to hormone ablation therapy. Clearly, novel treatment modalities for prostate cancer must be developed that can effectively target androgen-independent and androgen-dependent prostate cancers.

### SUMMARY OF THE INVENTION

In accordance with the present invention, a method for synergistically inducing apoptosis in cancer cells in a patient in need thereof is provided. An exemplary method entails administering an effective amount of a PI3K inhibitor and a toxin molecule, in a pharmaceutically acceptable carrier to a patient, the PI3K inhibitor and toxin molecule acting synergistically to rapidly induce apoptosis in a targeted cancer cell. The method optionally comprises administration of a chemotherapeutic agent or an agent conventionally used to treat prostate cancer. In a preferred embodiment, the PI3K inhibitor is selected from the group consisting of LY294002 and biologically active derivatives thereof, LY292223, LY293696, LY293684, LY293646, wortmannin, PX-866, ZSTK474, SF1126, BEZ235, VQD-002, KRX-0401, GSK690693 and XL147 and prodrugs thereof and the toxin is selected from the group consisting of Pseudomonas exotoxin (PE) A, PE40, ricin, ricin A-chain, diphtheria toxin, abrin, abrin A chain, modeccin A chain, alpha-sarcin, gelonin, mitogellin, retstrictocin, phenomycin, enomycin, curicin, crotin, and calicheamicin and prodrugs thereof. In a particularly preferred embodiment the cancer is prostate cancer, the PI3K inhibitor is a prodrug of LY294002 or ZSTK474 comprising a PSA cleavable linker and the toxin is Pseudomonas exotoxin (PE) A or PE40 operably linked to an antibody thereby forming an immunotoxin which has binding specificity for an antigen present on a prostate cancer cell. In certain embodiments, the immunotoxin optionally comprises a PSA cleavable linker. The prostate antigen is selected from the group consisting of PMSA, PCA, MUC1, Epidermal growth factor receptor, platelet-derived growth factor, platelet-derived growth factor receptor, urokinase plasminogen activator, and urokinase plasminogen activator receptor, with PMSA being particularly preferred.

In yet another aspect, the inhibitor and the toxin are each operably linked to an antibody immunologically specific for a prostate cell, thereby enhancing prostate cancer cell targeting. The inhibitor and toxin may be linked to the same or a different antibody.

In another embodiment of the invention, a synergistic anti-prostate cancer formulation is provided. Any exemplary formulation comprises, i) a LY294002 prodrug or ZSTK474 prodrug operably linked to a PSA cleavable linker which is effective to inhibit PI3K activity; and ii) a Pseudomonas exotoxin (PE) A or PE40 operably linked to an antibody which has binding specificity for PMSA antigen thereby forming an immunotoxin, said immunotoxin optionally comprising a PSA cleavable linker, each of i) and ii) being present in a pharmaceutically acceptable carrier. In another aspect, each of the inhibitor and toxin are linked to an antibody which has binding affinity for a prostate cancer cell.

### BRIEF DESCRIPTION OF THE DRAWINGS

FIG. 1. PI3K integrates signals from GPCRs, receptor tyrosine kinases and cytoplasmic tyrosine kinases. PTEN inhibits PI3K signaling. Loss of PTEN leads to constitutive activation of PI3K in the absence of upstream stimuli.

FIG. 2. Dual targeting increases concentration of active drug in the tumor. FIG. 2A shows PSA-activated PI3K pro-drug evenly distributed, but active only in tumor. FIG. 2B illustrates that antibody-targeted pro-drug accumulates in tumor and in liver, but activated only in tumor.

FIG. 3. A schematic of the anti cancer agents of the invention is shown. While ZSTK474 is depicted, any PI3K inhibitor which may be operably linked to the linker (e.g., LY294002 and/or the immunotoxin can be utilized.

FIG. 4 Injections of LY294002 inhibit PI3K activity and tumor growth. FIG. 4A) Representative luminescent images (superimposed on black and white images) of mice injected with solvent (DMSO) or LY294002. Injection site is indicated by arrow. FIG. 4B) Changes in luminescence of subcutaneous xenograft tumors injected with LY294002 or DMSO (error bars show standard deviations between 3 tumors injected with each agent). FIG. 4C) Tissue sections of xenograft tumors. Arrowheads point at apoptotic cells. FIG. 4D) Inhibition of Akt phosphorylation in xenografts injected with LY294002. Western blotting of tumor lysates.

FIG. 5. J591 antibodies recognize C42Luc cells. Indirect immunofluorescent staining of (A) C42Luc cells that express PSMA and (C) PC3 cells that do not express PSMA with J591 antibodies followed by FITC-labeled goat anti-mouse antibodies. Nuclei were visualized by DAPI. (B, D) Phase-contrast images of the same fields as (A, C) respectively.

FIG. 6. Administration of a PI3K inhibitor and a PT results in synergistic induction of apoptosis. Prostate cancer C42 cells were treated with 10 nM of TGFα-pseudomonas exotoxin chimera (PE) and 500 nM PI3K inhibitor ZSTK474 (ZSTK) or PI3K inhibitor LY294002 (LY) individually and in combination. A) Caspase activity was measured 3 h and 6 h after treatments began; B) Time lapse video-recording of C42 cells treated with 10 nM PE or combination of 10 nM PE and 500 nM ZSTK474. Representative images taken 2, 5, and 14 h after treatments began; C) Caspase activity measured 6 h after cells were treated with increasing concentrations of PE and 25 mcM of LY294002 (LY).

### DETAILED DESCRIPTION OF THE INVENTION

Existing therapies for prostate cancer invariably lead to the emergence of resistant cancer cells. Targeting such cells for apoptosis provides an effective approach for the treatment of cancer. We have discovered that exposing prostate cancer cells to a combination of phosphatidylinositol-3-kinase (PI3K) inhibitors and a toxin molecule such as pseudomonas exotoxin results in a synergistic induction of apoptosis in targeted prostate cancer cells.

Thus, in accordance with the present invention a method for treating prostate cancer is provided which entails the administration of a PI3K inhibitor which may be active per se or in the form of a pro-drug in combination with a toxin which comprises a prostate cell targeting moiety which selectively binds prostate cells which may also optionally be in the form of a pro-drug.

### DEFINITIONS

“Prostate cancer” refers to the presence of malignant cells in the prostate. The terms “advanced prostate cancer”, “locally advanced prostate cancer”, “advanced disease” and “locally advanced disease” mean prostate cancers that have extended through the prostate capsule, and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asymmetry or induration above the prostate base. Locally advanced prostate cancer is presently diagnosed pathologically following radical prostatectomy if the tumor invades or penetrates the prostatic capsule, extends into the surgical margin, or invades the seminal vesicles.

As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis.

As used herein, the phrase “effective amount” of a compound or pharmaceutical composition refers to an amount sufficient to modulate tumor growth or metastasis in an animal, especially a human, including without limitation decreasing tumor growth or size or preventing formation of tumor growth in an animal lacking any tumor formation prior to administration, i.e., prophylactic administration.

The term “mammal” refers to both animals and humans.

As used herein, the phrase “phosphatidylinositol-3-kinase (PI3K) inhibitor” refers to an agent which is effective to inhibit PI3K activity. Agents which inhibit the β isoform of PI3K are particularly preferred. Exemplary agents include LY294002 and biologically active derivatives thereof, LY292223, LY293696, LY293684, LY293646 (Vlahos et al. J. Biol. Chem. 269:5241-5248 (1994), wortmannin (Sigma-Aldrich), PX-866, a wortmannin derivative in Phase I clinical trials (Oncothyreon) ZSTK474 (Zenyaku Kogyo Co.), SF1126 (Semaphore Pharmaceuticals), BEZ235 (Novartis) VQD-002 (VioQuest Pharmaceuticals) KRX-0401 (Keryx Biopharmaceuticals) GSK690693 (GlaxoSmithKine), XL147 (Exelixis) and siRNA and shRNA molecules which specifically hybridize with PI3K beta encoding mRNA and interfere with intracellular production thereof. In a preferred embodiment, the PI3K inhibitor is a prodrug of LY294002 or ZSTK474 comprising a prostate specific antigen cleavable linker which is activated at the site of the prostate cancer cell. This prodrug is administered in combination with a toxin comprising a cancer targeting moiety such as an antibody, or an immunospecific fragments thereof.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668). Antibodies immunospecific for antigens present on prostate cells are particularly preferred for use in the present invention. Such antigens include, without limitation, PMSA, PSCA, MUC1, Epidermal growth factor receptor, platelet-derived growth factor, platelet-derived growth factor receptor, urokinase plasminogen activator, and urokinase plasminogen activator receptor.

A “toxin” refers to a substance that inhibits or prevents the expression activity of cells, function of cells and/or causes destruction of cells. The term is intended to include small molecule toxins or enzymatically active toxins of bacterial, fungal, plant or animal origin, including fragments and/or variants thereof which are effective to inhibit protein synthesis in a target cell. Examples of toxins include, but are not limited to Pseudomonas exotoxin (PE) A, PE40, ricin, ricin A-chain, diphtheria toxin, abrin, abrin A chain, modeccin A chain, alpha-sarcin, gelonin, mitogellin, retstrictocin, phenomycin, enomycin, curicin, crotin, and calicheamicin. Antibodies may also be conjugated to such toxins, thereby forming an “immunotoxin” to facilitate targeting to the prostate cancer cell. Such immunotoxins may also be generated as prodrugs which comprise an operably linked PSA cleavable peptide which masks the antigen binding site until cleaved by PSA elaborated by prostate cancer cells.

As used herein, the term “prodrug” refers to a precursor form of the drug which is metabolically converted in vivo to produce the active drug. Thus, for example, PI3K inhibitors, (e.g., LY294002 and ZSTK474) in the form of prodrugs are administered to an mammal in accordance with the present invention which undergo subsequent metabolic activation and regenerate active LY294002 or ZSTK474 at the site of interest (e.g., at the prostate) in vivo, e.g., following exposure to endogenous PSA protease in the body.

A “linker” refers to a peptide sequence which can linked to a drug of interest (e.g., a PI3K inhibitor) and be activated on by proteases expressed by targeted cells (e.g., PSA from prostate cells). Preferred linker molecules for use in the present invention include, without limitation, HSSKLQL (SEQ ID NO: 1), CHSSKLQG (SEQ ID NO: 2); EHSSKLQ (SEQ ID NO: 3), QNKISYQ (SEQ ID NO: 4), INKISYQ (SEQ ID NO: 5) and ATKSKQH (SEQ ID NO: 6 (SEQ ID NO: 6).

In certain embodiments, it may be desirable to include administration of at least one additional chemotherapeutic agent with the compositions described herein. Suitable chemotherapeutic agents include, but are not limited to: alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In a particular embodiment, the chemotherapeutic agent is selected from the group consisting of: placitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

Agents typically employed for the treatment of prostate cancer may also be utilized in the methods of the present invention. Such agents include, without limitation, Casodex®, hormone ablating agents, Lupron®, radiation, radioactive seed implantation and gamma knife surgery.

As used herein, the phrase “sub-therapeutic dose” refers to dosage levels which are lower than those typically employed to treat disease. Because the compounds of the invention work in a synergistic manner, lower than normal doses can be employed thereby diminishing side effects associated therewith.

### Pharmaceutical Compositions

As explained above, the present methods can, for example, be carried out using a single pharmaceutical composition comprising both LY294002 compound or a derivative thereof and PT (when administration is to be simultaneous) or using two or more pharmaceutical compositions separately comprising the PI3K inhibitor compound and toxin(s) (when administration is to be simultaneous or sequential). Such pharmaceutical compositions also comprise a pharmaceutically acceptable carrier. The phrase “pharmaceutically acceptable” refers to molecular entities and compositions that are physiologically tolerable and preferably do not produce an allergic or similar untoward reaction, such as gastric upset, dizziness and the like, when administered to a human.

Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A pharmaceutical composition of the present invention can be administered by any suitable route, for example, by direct injection, intravenous infusion, or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

The PI3K inhibitor and toxin may be employed in any suitable pharmaceutical formulation, as described above, including in a vesicle, such as a liposome [see Langer, Science 249:1527-1533 (1990); Treat et al., in Liposomes in the Therapy of Infectious Disease and Cancer, Lopez-Berestein and Fidler (eds.), Liss: New York, pp. 317-327, see generally, ibid] Preferably, administration of liposomes containing the agents of the invention is parenteral, e.g., via intravenous injection, but also may include, without limitation, intra-arteriole, intramuscular, intradermal, subcutaneous, intraperitoneal, and intraventricular, administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudtk et al., N. Engl. J. Med. 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115-138 (1984)]. In particular, a controlled release device can be introduced into an animal in proximity of the site of inappropriate immune activation or a tumor. Other controlled release systems are discussed in the review by Langer (Science 249:1527-1533 (1990).

The following materials and methods are provided to facilitate the practice of the present invention.

Cell lines—Prostate Cancer cell lines, LNCaP and C4-2, were gifts from Dr. Leland Chung (Emory University, Atlanta Ga.). C4-2Luc cells were generated by transfecting C4-2 cells with pTRE2hygro and firefly luciferase (PGL3). C4-2 cells were maintained with RPMI 1640 with 10% fetal bovine serum. All cells were kept at 5% CO₂ at 37° C.

Antibodies and Other Reagents—Antibodies were obtained from the following sources: Akt, phospho-specific Akt (S473, T308) from Cell Signaling Technology (Beverly, Mass.); J591 (a single chain antibody immunospecific for PSMA was provided by Dr Bander (Weil Medical College, NY, N.Y.). Tissue culture reagents were purchased from Invitrogen (Carlsbad, Calif.).

Western blotting—Tumors were excised, homogenized on ice in lysis buffer [10% glycerol, 50 mM PIPES-KOH (pH 7.4), 5 mM EDTA, 1% NP40, 1 mM DTT, protease and phosphatase inhibitors;] centrifuged 10 min 14,000 G to precipitate unsoluble fraction. Soluble fraction was resolved by SDS-PAGE followed by Western blotting on nitrocellulose. Nitrocellulose membranes were probed with antibodies to Akt and phospho-Akt according to manufacturer's instructions; the appropriate secondary antibodies were then applied and positive signals detected by enhanced chemiluminescence.
Immunofluorescence—Cells were plated on glass coverslips in 6-well plates and treated and fixed with 10% formalin in PBS (pH 7.4) for 15 min. The cells were then blocked in 2.5% goat serum in PBS-T for 30 min at 37° C. Blocking solution was used to prepare a 1:300 mixture of mouse-J591 anti-PSMA antibodies subsequent procedures were done at room temperature. Incubation with primary antibodies was continued for 3 h. The cells were then washed three times with PBS-T and incubated with a mixture of goat antimouse antibodies conjugated with fluorescein, diluted in blocking solution at a ratio of 1:300 each. One h later, cells were washed with PBS-T, stained with DAPI, and mounted on glass microscope slides. Image analysis was done on a Nikon microscope equipped with a digital camera and software from Inovision (Durham, N.C.).
Tumor Implantations—Nude mice (BALB/cAnNCrj-nu from Charles River) received four subcutaneous injections or two intratibial injections of 2×10⁶ cells with Matrigel. Injections were made using an insulin syringe and a 27 gauge needle. All manipulations with animals were conducted in humane manner, in strict adherence with a protocol approved by institutional ACUC, which was designed to minimize animal suffering.
Luminescence Imaging—Tumor growth was analyzed with a Xenogen IVIS® 100 optical imaging system (Caliper Life Sciences, Hopkinton, Mass.). Animals were immobilized for substrate injection and imaging through an attached gas anesthesia system consisting of 2% isoflurane/O₂. To account for background and nonspecific luminescence, mice were imaged before injection of luciferase. Animals were injected with 100 μl of the firefly luciferase substrate luciferin (3.5 mg/ml in PBS) and imaged 15 minutes later in prone and supine positions (5 minutes each). Whole-body images were obtained using the Living Image® software provided with imaging system. A gray-scale photographic image and the bioluminescent color image are superimposed to provide anatomic registration of the light signal. A region of interest (ROI) was manually selected over the luminescent signal, and the intensity was recorded as photons/second within an ROI.
Apoptosis Assays—Apoptosis in whole cell populations was quantified by measuring caspase-3 activity with the fluorogenic substrate Ac-DEVD-7-amido-4-trifluoromethyl-coumarin (Bachem) as specified by the manufacturer. For these experiments, attached and floating cells were collected and lysed in caspase lysis buffer (1% Nonidet P-40, 150 mM NaCl, 20 mM HEPES, 1 mM EDTA, 1 mM dithiothreitol, and 5 μg/ml aprotinin, leupeptin, and pepstatin). Fluorescence was recorded each 15 min for 1 h, and caspase activity was expressed in arbitrary units. Apoptosis was also measured by time lapse video recording followed by counting the percentage of cells with apoptotic morphology (assessed as cytoplasmic blebbing and fragmentation). At least four randomly chosen fields (containing on average 200-300 cells for each treatment) were recorded. Video recording was performed on an Axiovert100 microscope (Carl Zeiss, Germany) equipped with a moving stage and climate control chamber (37° C., 5% CO₂) and controlled by Openlab software (Improvision Inc., Lexington, Mass.). The results reported herein were confirmed by at least two independent experiments.
Statistical analysis—To determine whether differences between data sets were statistically significant, Student's t-test analysis (two-tailed distribution; two-sample unequal variance) was performed using Excel software.

The following example is provided to illustrate certain embodiments of the invention. They are not intended to limit the invention in any way.

### Example I

### Synergistic Induction of Apoptosis in Prostate Cancer Cells and Synthesis of Efficacious Pro-Drugs for Use in Combination

Androgen ablation therapy introduced by Charles Huggins in 1941 remains the most effective systemic treatment for prostate cancer. In most cases, the disease initially responds to androgen ablation therapy. However, it eventually recurs as androgen-independent prostate cancer, for which no effective treatment is currently available (Crawford et al. (1989) N. Engl. J. Med. 321:419-424; Denis et al. (1993) Cancer 72:3888-3895). Normal prostate epithelial cells undergo apoptosis after androgen levels are decreased (Kyprianou et al. (1988) Endocrinology 122:552-562). Thus, apoptosis is a default pathway for normal prostate cells after androgen withdrawal. It was proposed that in order to survive, androgen-independent prostate cancers activate the androgen-independent mechanisms that inhibit apoptosis (Dorkin et al. (1997) Semin. Cancer Biol. 8:21-27, Tang et al. (1997) Prostate 32:284-293).

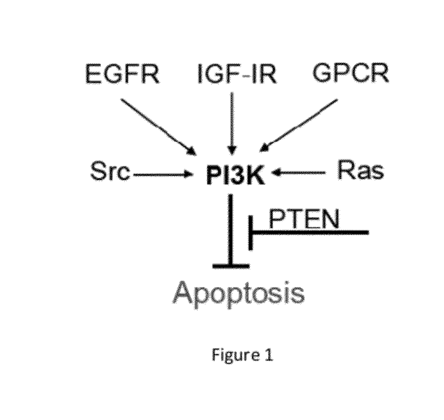

Activation of PI3K signaling occurs when external growth factors trigger recruitment of PI3K to the plasma membrane, where it phosphorylates phosphatidylinositol at the 3-d position. Phosphatidylinositol 3 phosphate in turn engages serine/threonine protein kinases like PDK1 and Akt through binding to their PH domains, as well as other kinases that send numerous downstream signals that regulate cell metabolism, cell division and survival (Vivanco et al. (2002) Nat. Rev. Cancer 2:489-501). PI3K signaling is negatively regulated by the lipid phosphatase PTEN, which dephosphorylates phosphatidylinositol 3 phosphate (FIG. 1). Loss of PTEN phosphatase is one of the most common intrinsic mechanisms of constitutive activation of PI3K signaling in cancer cells (Whang et al. (1998) Proc. Natl. Acad. Sci. 95:5246-5250).

Data from analyses of clinical samples and established prostate cancer cell lines have shown that substantial proportion of prostate cancers rely on PI3 kinase signaling for their growth and survival. Indeed, the loss of PTEN phosphatase has been documented in 30% of primary and 60% of androgen-independent metastatic cancers (Vivanco et al, supra). A prostate-restricted knockout of PTEN in mice is sufficient to trigger the development of metastatic prostate cancer (Wang et al. (1998) Cancer Cell 4:209-221).

Constitutive activation leads to “addiction” of cancer cells to the PI3K pathway. As a result, inhibition of PI3K sensitizes prostate cancer cells to apoptosis (Kulik et al. (2001) Cancer Res. 61:2713-2719).

### Synergy Between PI3K Inhibitors and Other Cytotoxic Drugs.

Our recent experiments have shown that combination of PI3K inhibitor and other cytotoxic drugs like thapsigargin or Pseudomonas exotoxin A induces massive apoptosis in prostate cancer cells within 6 hours, whereas when these agents are applied individually apoptosis is induced much slower—within 12-24 hours. Thus, methods are provided for combined administration of a PI3K inhibitor with Pseudomonas exotoxin A to achieve rapid induction of apoptosis and tumor regression. Comparison of PI3K inhibitors LY294002, BEZ235 and ZSTK474 has shown that maximal caspase activation is achieved at 25 μM, 5 μM and 0.5 μM respectively; indicating that ZSTK474 is a potent inducer of apoptosis.

According to published in vivo experiments 80 nM of recombinant 40 kD c-terminus of Pseudomonas exotoxin A (PE40) fused to anti-PSMA antibodies inhibits C42 cell growth by 90%. In our experiments, maximal induction of apoptosis by combination of PI3K inhibitor (25 μM LY294002 or 0.5 μM of ZSTK474) and 10 nM of TGFαPE40 fusion was observed. Thus, in one aspect, the invention entails administration of prostate-targeted PE40 and ZSTK474 or LY294002 in order to synergistically induce apoptosis in prostate cells.

Drug Combination that Synergistically Induces Apoptosis in Targeted Cells.

In order to spare normal tissues from toxic side effects, it is necessary to design a drug delivery approach that selectively increases concentration of active drug in the tumor. One of the earliest approaches for increasing tumor-specificity, entails fusing cytotoxic drugs with ligands that bind tumor specific targets. Examples include ligands of EGFR that often overexpressed in cancer cells and antibodies to tumor-specific antigens like prostate specific membrane antigen (PSMA). To be suitable for tumor-targeting strategy drugs should be potent enough to kill tumor cells at nanomolar concentrations. Bacterial toxins and high energy radionuclides satisfy these criteria and have been tested successfully in mouse models of cancer. Another approach is to take advantage of enzymes exhibiting elevated activities in tumors. Both normal and malignant prostate cells secrete PSA, a protease with chymotrypsin-like substrate specificity. PSA is proteolytically active in the extracellular fluid of prostate cancers, but inactive in circulation, where it forms a complex with the serum protease inhibitors α-1-antichymortrypsin and α-2-macroglobulin (Denmeade et al. (2003) J. Natl. Cancer. Inst. 95:990-1000). In 1994, investigators from John Isaacs' laboratory proposed a pro-drug approach based on coupling thapsigargin to a peptide carrier via the PSA-cleavable peptide bond HSSKLQ (SEQ ID NO: 1). The peptide prevented the thapsigargin pro-drug from entering cells, and therefore rendered it non-toxic (Denmeade et al., supra). Other groups developed PSA-activated pro-drugs by coupling a PSA substrate peptide to 5-fluorodeoxyuridine (FudR), doxorubicin, vinblastine, proaerolysin and pro-apoptotic BH3-mimetic peptides (Khan et al. (2000) Prostate 45:80-83). In summary, the PSA-activated pro-drug approach has been used to modify compounds that are otherwise toxic to any cell.

However despite substantial enrichment of drug concentration in the tumor, off target toxicity remained a formidable problem. Thus, PSMA-directed antibody-toxin conjugates show kidney and liver toxicity due to increased PSMA expression in kidney and clearance of antibodies by the liver. In case of PSA-activated pro-drugs, “nonspecific” toxicity was attributed to non-specific protease activity resulting in cleavage of the PSA substrate at sites other than prostate, albeit with somewhat lower potency. As a result, “enrichment” in the tumor area was only four-fold.

Anti-cancer drugs have to be present at cytotoxic concentrations for a substantial length of time (12-24 h) to achieve anti-tumor effects. Despite improved tumor targeting, maintaining sufficient concentrations of cytotoxic drugs for the requisite time periods is problematic since “active” drugs diffuse out of tumor area and cause side effects.

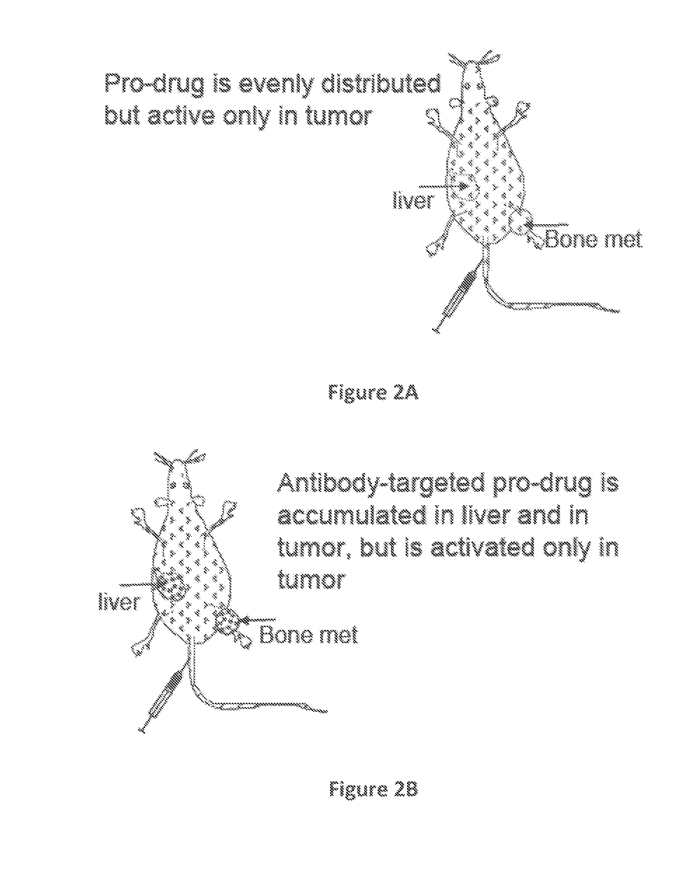

To avoid these drawbacks, we have combined two agents, PSA-activated PI3K inhibitor pro-drug with Pseudomonas exotoxin A conjugated to antibodies against prostate-specific membrane antigen (PSMA) in order to synergistically and rapidly induce apoptosis in prostate cancer cells. Monoclonal antibodies against the extracellular domain of PSMA (J591) have been used successfully to deliver toxins and radioisotopes to metastatic prostate tumors (Ross et al. (2005) Cancer Metastasis Rev. 24:521-537). Because antibodies accumulate in liver, the main limitation of antibody-directed therapy is liver toxicity (Ross et al., supra). We anticipate that using PSA-activated pro-drug (instead of active inhibitor) coupled to J591 antibody will substantially reduce toxic effects outside of the prostate tumor. At the same time prostate tumor targeting with J591 antibodies will increase pro-drug concentration in the tumor and thus will help to achieve therapeutic concentration of active inhibitor (FIG. 2). We anticipate that this strategy will achieve anti-tumor effect that exceeds antitumor effects observed with single cytotoxic agents targeted by tumor-specific ligands or PSA cleavage.

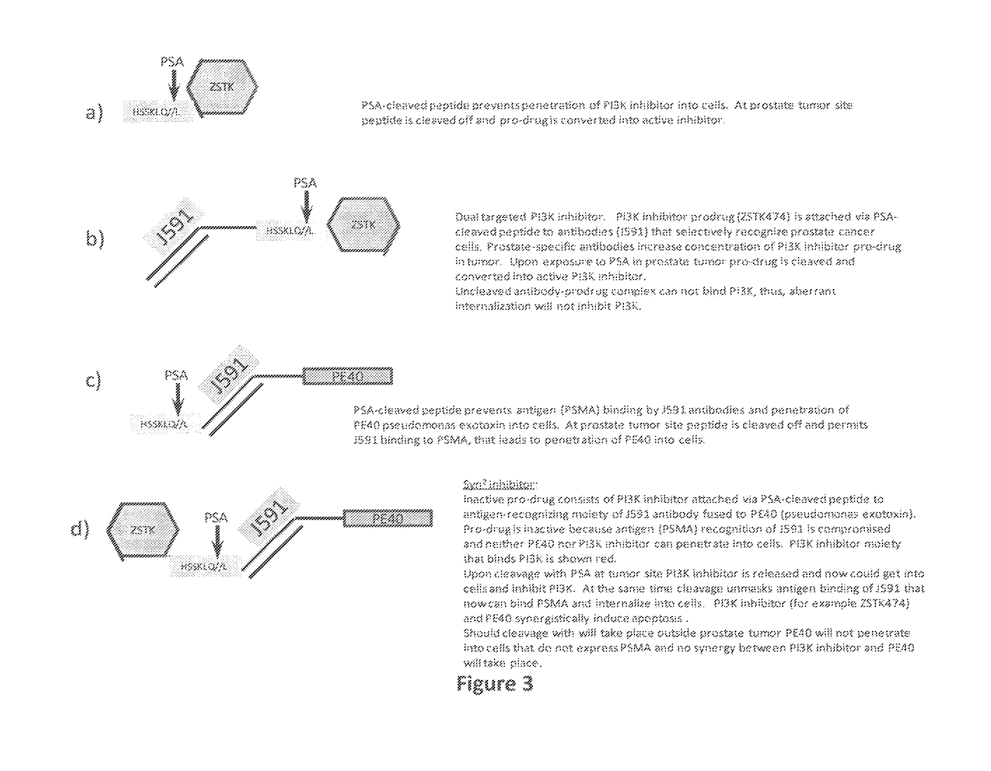

The anti-cancer prodrugs described herein have several components (FIG. 3). The PI3K inhibitor is formulated as a prodrug comprising a PSA cleavable linker (FIG. 3a). The inhibitor may further comprise an antibody targeting moiety, such as J591 which is immunospecific for PMSA (FIG. 3b). In this embodiment, the uncleaved antibody-prodrug complex cannot bind PI3K until elaborated at the tumor site following PSA mediated cleavage. In yet another embodiment, the immunotoxin comprises a PSA cleavable linker thereby which masks antigen binding until the linker is cleaved at the prostate tumor site (FIG. 3c). A Syn2 inhibitor is shown in FIG. 3d wherein the immunotoxin and PI3K inhibitor prodrug are each operably linked to an antibody having immunospecificity for an antigen present in prostate cancer cells.

### Testing Tumor-Targeted PI3K Inhibitors in the Model of Metastatic Prostate Cancer

Up to 70% of men who die of prostate cancer have bone metastases—a major cause of suffering and mortality (Catalona et al. (1994) N. Eng. J. Med. 331:996-1004). Therefore, experimental therapies for prostate cancer would be especially valuable if they show efficacy in models of androgen-independent metastatic prostate cancer. One obstacle in this area is the technical difficulty of modeling metastatic prostate cancer in experimental animals. Genetically modified mice that develop prostate cancer rarely present with metastases because they die from bladder obstruction before metastases occur.

We will model bone metastases by implanting C42 cells into the femur of SCID mice. This approach results in obtaining tumors in bones within two months of implantation with high frequency (Corey et al. (2002) Prostate 52:20-33). The C42 cells do not express PTEN due to frame-shift mutation, and therefore the PI3K pathway is constitutively active in these cells. C42 cells retain the expression of androgen receptor and respond to androgen. However, they form xenograft tumors in castrated mice and maintain PSA expression in the absence of androgen (Thalmann et al. (2000) Prostate 44:91-103). Thus, this model is appropriate to examine effects of PI3K inhibitors on androgen-independent metastatic prostate cancer.

We have generated C42Luc cells that stably express firefly luciferase. C42Luc cells form xenograft tumors when implanted in the tibia, femur, or subcutaneously, and growth of these xenografts can be followed by noninvasive luminescent imaging on an IVIS station. Luminescent imaging allows us to detect and quantify tumors growing internally in living mice with high sensitivity.

### Synthesis of PI3K and Toxin Pro-Drugs

A variety of agents are available which exhibit the capacity to inhibit PI3 kinase. Several of these agents are listed above. LY294002 is particularly well known for this purpose. Pilot data from computer modeling indicates that the para position of the unfused benzene ring (labeled in structures 1-3 below with an X) allows attaching of linkers without compromising interactions of LY294002 with the ATP binding site of PI3 kinase.

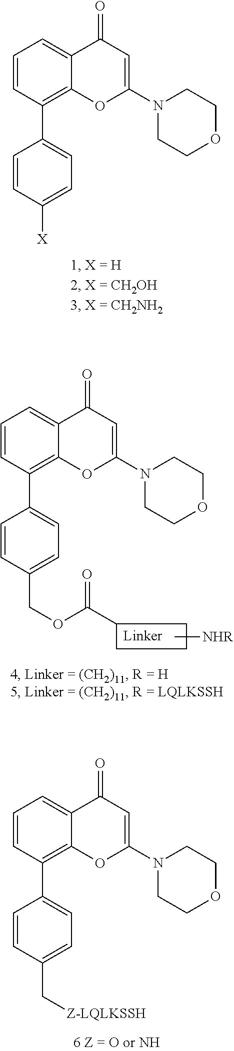

Chemical syntheses will be conducted as follows: We will first synthesize analogs of LY294002 (1) which contain functional groups which should not adversely affect their PI3K binding ability (Walker et al. (2000) Mol. Cell. 6:909-919) but will permit the attachment of fatty acid linkers or the direct attachment of peptides (compounds 2 and 3). Synthesis of analogs 2 and 3 should be straightforward and will be modeled after the synthetic strategy presented by Abbott and Thompson (Aust. J. Chem. 2003, 56, 1099-1106). Commercially available 2,3-dihydroxybenzoic acid (7) will first be converted to the methyl ester (8). The least sterically hindered hydroxyl will next converted to a triflate by treatment with trifluoromethyl sulfonic anhydride (Tf20, triflic anhydride) to produce 9. The enolate of N-acetyl morpholine (10) will then generated with lithium diisopropyl amide (LDA) and condensed with the ester in 9 to produce 11. Treatment of 11 with triflic anhydride will induce cyclization to 12. The triflate will then be subjected to Suzuki coupling with 2 commercially available boronic acids. Coupling with 4-(hydroxymethyl)phenyl boronic acid (13, X═CH2OH) will produce the LY294002 analog with the CH₂OH functional group (2), whereas coupling to 4-(aminomethyl)phenyl boronic acid (13, X═CH2NH2) will produce 3.

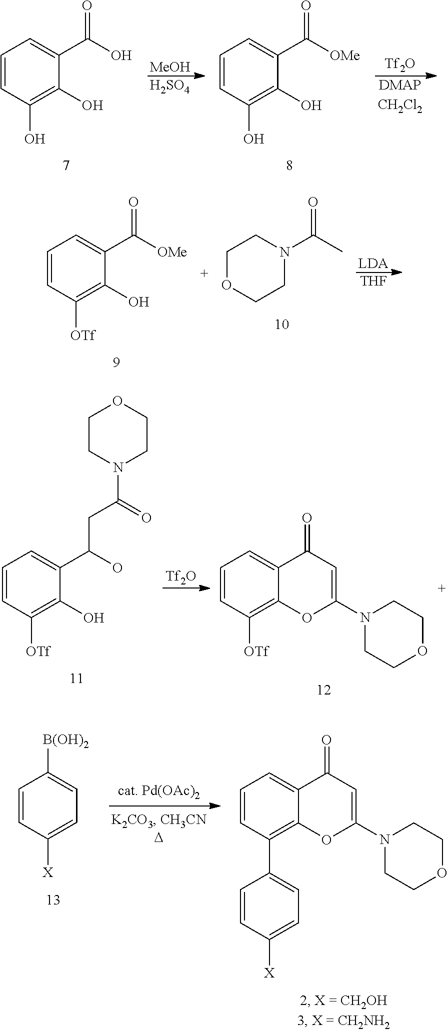

Once the LY294002 analogs with primary alcohol and amine functional groups are prepared, two options will exist for attaching the PSA-cleavable HSSKLQL peptide chain to these molecules. The first option would involve preparation of Boc protected 12-[L-leucinoylamino]dodecanoic acid (17), as described by Christensen and co-workers (Jakobsen et al. (2001) J. Med. Chem. 44:4696-4703, and coupling it to 2, followed by TFA removal of the protecting group to produce 4.

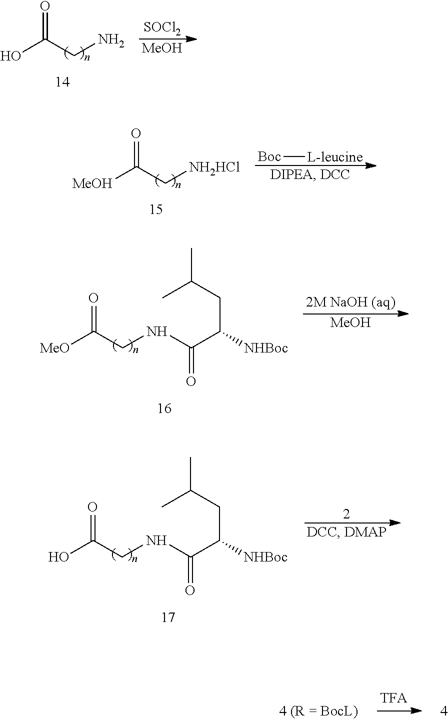

Compounds 4 shown above will be screened as PI3K inhibitors. The activity of compound 4 will be critical, since this fragment would remain once PSA has cleaved the HSSKLQ peptide off the pro-drug. If the activity of 4 is affected by the fatty acid linker, we can try other shorter and more aromatic linkers, as described by Christensen and co-workers (Jakobsen et al., supra).

The second option would be synthesize the compound without the fatty acid linker, where 2 is directly attached to Boc protected leucine (2-L). The Boc protecting group can be removed by TFA when desired. This same strategy will also be followed initially with the amine analog of 2, compound 3. Compound 3 will also be attached to the fatty acid linker containing a terminal L as described above. If the fatty acid linker affects its activity, then direct attachment of 3 to L as described below would yield 3-L. Compounds 2-L and 3-L represent what would remain after PSA have cleaved off substrate peptide from pro-drug 6.

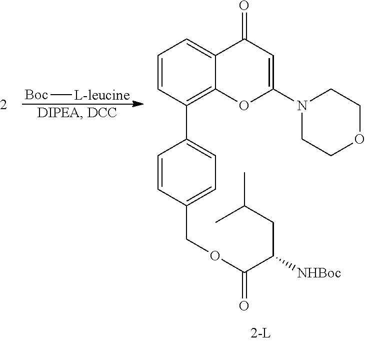

Final products will be purified by HPLC and their ability to inhibit PI3K activity confirmed.

PI3K inhibitor pro-drugs will be made by attaching a PSA-cleavable peptide to the modified LY294004 analog described above. Tissue culture experiments will be conducted to confirm that pro-drugs can not inhibit PI3K. Then, PI3K inhibitor pro-drugs will be treated with PSA to demonstrate that removing the PSA-cleavable peptide will convert pro-drug into active PI3K inhibitor.

Thus, the following steps will be carried out: 1) inactive pro-drug will be synthesized by attaching the HSSKLQ peptide substrate of PSA to L-LY294002 analog synthesized and tested as above; 2) Activation of the HSSKLQL-LY294002 pro-drug by PSA secreted by C42Luc cells will be assessed as will inhibition of PI3 kinase activity and induction of apoptosis.

PSA-cleavable peptide HSSKLQ (SEQ ID NO: 1) will be attached to C-terminus of Pseudomonas exotoxin conjugated with anti-PSMA antibodies A5-PE40. The resulting pro-toxin will be tested in tissue culture to confirm that it is inactive. Then, pro-toxin will be incubated with PSA to test whether removing HSSKLQ peptide will restore cytotoxic effect. Unmodified A5-PE40 or TGFα-PE40 will be used as positive control.

As mentioned above, ZSTK474 is a potent inhibitor of PI3K and thus can also be used to advantage in the methods described herein. As above, it certain embodiments, it is desirable to generate prodrugs of ZSTK474 which are activatable at the site of the prostate tumor.

ZSTK474 (18) is known to inhibit PI3K more efficiently than LY294002 (J. Natl. Can. Inst. 2006, 98, 545-556) and ZSTK474 contains one aromatic ring where a primary alcohol or primary amine linkers couple be attached which could also be used as chemical attachment points (19, 20; X═OH or NH₂).

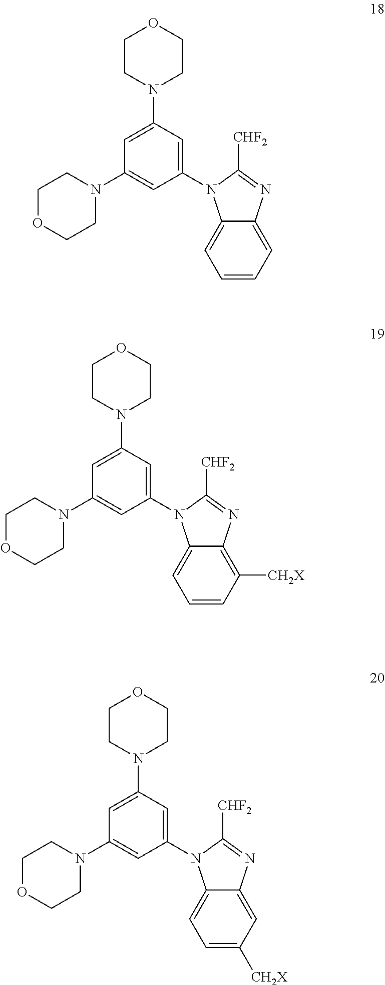

ZSTK474 is prepared by sequential addition of morpholine (22), the benzimidazole (23), and then morpholine again to commercially available cyanuric chloride (21) (Chem. Pharm. Bull. 2000, 48, 1778-1781). Adapting this synthetic scheme to the production of 19 and 20 then boils down to the production of the appropriate benzimidazoles (24 and 26). While a CHF₂ group is shown in 18, 19 and 20, in an alternative scheme, this group is replaced with a CF₃ group. The benzimidazoles could be produced as a mixture of N—H tautomers (24 and 25) (26 and 27) but based on the literature, they should N-alkylate through the least sterically hindered tautomers (24 and 26) (Tet. Lett. 1988, 29, 3033-3037).

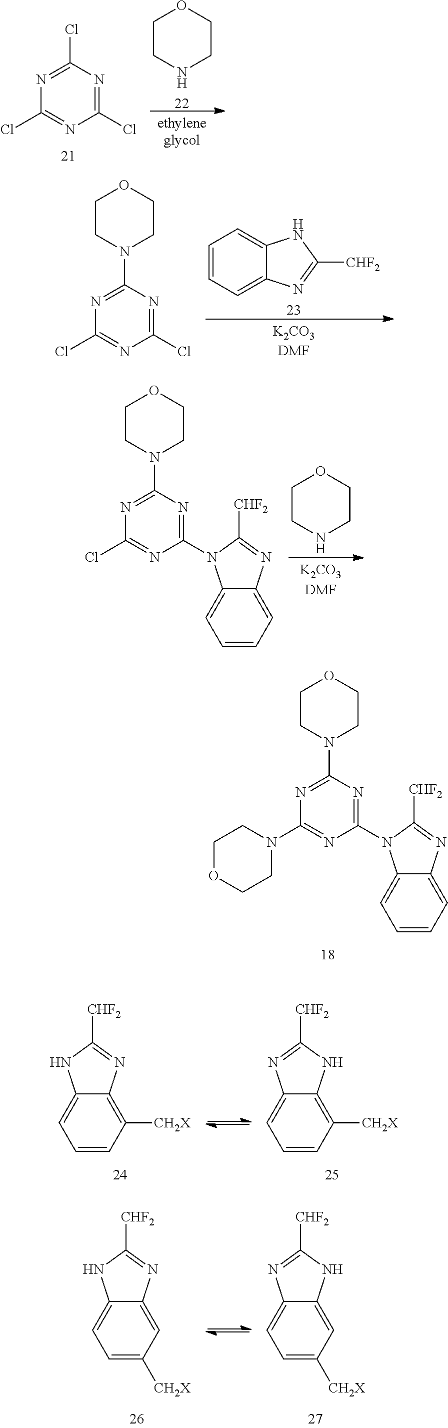

We have started these synthetic procedures initially aimed at the production of the primary alcohol substrates (24 and 26, X═OH). Production of those compounds involves conversion of the commercially available 2,3- or 3,4-diaminobenzoic acids (28) into their ethyl esters (29) followed by reduction of the esters to the primary alcohols (30) (Heterocycles, 2007, 71, 2491-2497) followed by condensation with the aldehyde to generate the benzimidazole (26 X═OH) (J. Heterocyclic Chem. 2008, 45, 1293-1298). Once the ZSTK474 analogs have been generated fatty acid linkers and the PSA cleavable peptide sequence can be attached as described above for LY 294002 analogs (4) or just directly attach leucine plus the PSA cleavable sequence as described above for LY294002 analogs (5).

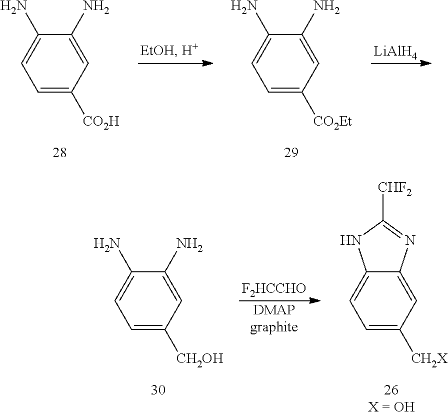

Analysis of apoptosis will be performed by caspase assays and time lapse video microscopy described previously (Sastry et al., (2006) J. Bio. Chem. 281:20891-20901.

Prior to experiments with tumor xenografts, the maximal tolerated dose (MTD) of PI3K inhibitor and A5-PE40 pro-drugs in immunocompromised (cAnNCr-nu) mice will be determined. To determine the maximum tolerated dose of Boc-HSSKLQL-LY294002 or a ZSTK474 prodrug, mice will be injected with a starting dose of the molar equivalent of 100 mg/kg LY294002 (400 mg/kg of pro-drug) and 0.6 mg/kg of A5-PE40 pro-drug with subsequent 3-fold escalations until toxic effects are detected. Each dose will be injected intraperitoneally in 3 SCID mice daily for 7 days. Dose escalation will be stopped if at least one mouse in a group dies. In subsequent experiments, we will use pro-drugs in concentrations that do not show adverse effects or reduce weight by more than 10%. Experiments will be conducted in mice implanted with C42Luc xenografts subcutaneously and into the femur. Subcutaneously implanted tumors can be conveniently followed and excised for analysis; however, they do not reflect localization of human prostate cancer. Thus, in addition to subcutaneous xenografts, experiments will be conducted on xenografts implanted into femur to model bone metastases. When subcutaneous tumors reach 5 mm diameter and intrafemoral tumors produce luminescence equivalent to a subcutaneous tumor 5 mm in diameter (105 ph/s/cm2/sr), mice will be castrated and randomly divided into experimental and control groups, with at least 5 mice in each. Growth of C42Luc xenografts is not inhibited by castration, however androgens may inhibit apoptosis and tumor regression induced by PI3K inhibitors. Then, mice will be intraperitoneally injected either with the Boc-HSSKLQL-LY294002 pro-drug (maximal tolerated dose, determined as described above), A5-PE40 pro-drug or with solvent. As positive control for proper tumor targeting pro-drugs will be injected directly into tumor xenografts.

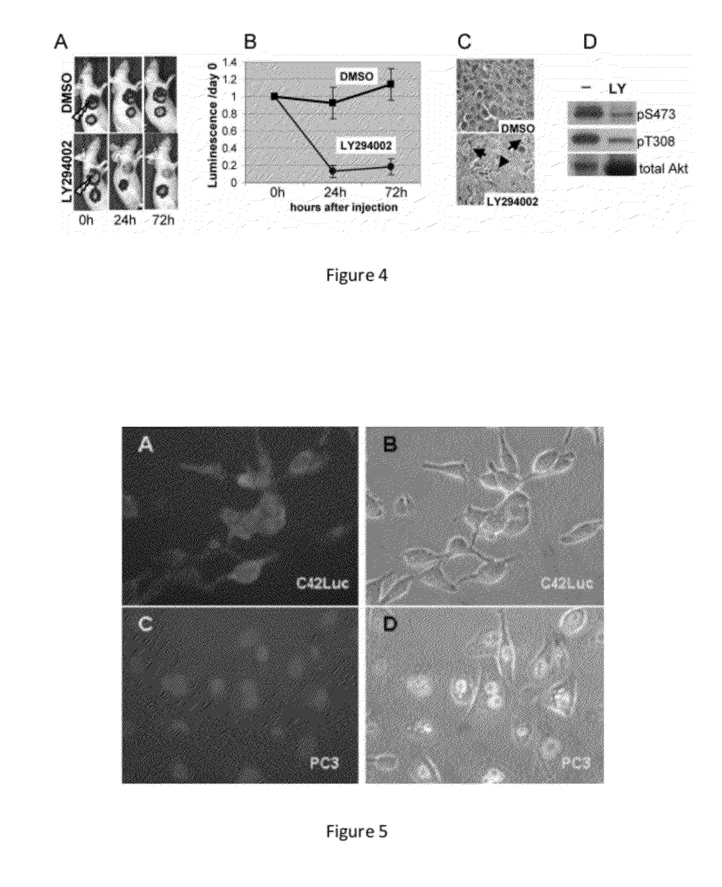

Effects on tumor growth will be determined by monitoring luminescence of C42Luc xenografts by optical imaging on an IVIS station. Luminescence will be recorded immediately before injections (day 0) and monitored for 3 weeks every two days. Effect on tumor growth will be expressed as fold change relative to luminescence on day 1. Based on pilot experiments (FIG. 4), we estimated that the standard deviation of the relative tumor luminescence values in the control group is approximately 10%; therefore, a 20% difference corresponds to a 2 standard deviation effect size. Using a 2-sample t-test, we will have 80% power to detect this difference between groups if we have n=5 animals per group, assuming a 2-sided test with alpha=0.05. Based on the differences between groups and level of standard deviations within groups injected with pro-drug or solvent, the number of mice in subsequent experiments may be adjusted to obtain statistically significant results. Also, we will test whether PI3K signaling in xenografts is inhibited and whether tumor cells undergo apoptosis. For this purpose, tumor tissue sections will be prepared from one part of xenografts and stained with antibodies against cleaved caspase 3. The second part of xenografts will be lysed and used to examine Akt phosphorylation and cleavage of PARP and caspases by Western blotting.

PSMA has been identified as a transmembrane protein preferentially expressed in prostate epithelial cells (Ross et al., supra). Since PSMA expression is preserved in prostate cancer cells, antibodies to PSMA have been successfully used to target anti-cancer drugs to prostate tumors. Thus, several radioisotopes and cytotoxins fused to J591 monoclonal antibodies against PSMA have demonstrated improved antitumor efficacy in xenografts (Ross et al., supra). Phase II clinical trials with J591 are currently ongoing. Pilot studies in patients show that despite an increase in drug concentration in the tumor, substantial quantities of J591 conjugates were accumulated in liver. As a result, liver toxicity could limit the use of antibody-targeted drugs against prostate tumors, as well as other tumors.

Because the HSSKLQL-LY294002 pro-drug is not expected to be activated outside of tumor, its liver toxicity should be much less then of conventional antibody-toxin complexes. At the same time, improved tumor targeting will increase pro-drug concentration in the tumor. An additional advantage of using J591 antibodies as the pro-drug targeting moiety is that several pro-drug molecules could be coupled to one antibody molecule. This may further increase local concentration of pro-drug outside tumor and also could permit attaching several synergistically acting pro-drugs. We have already demonstrated that J591 antibodies bind to C42Luc cells that express PSMA but not PC3 cells that do not express PSMA (FIG. 5).

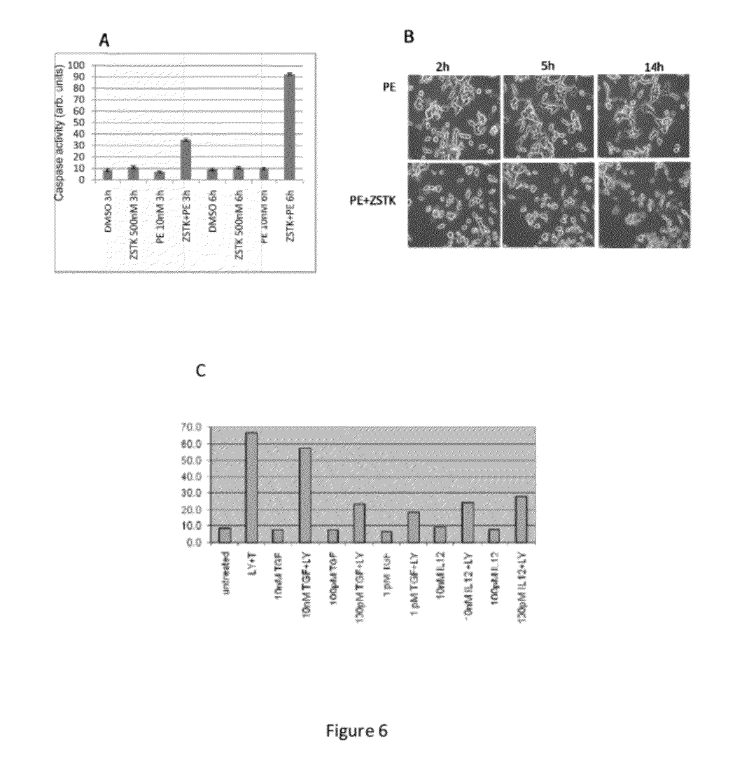

Synergistic induction of apoptosis by PE and two different PI3K inhibitors is shown in the data presented in FIG. 6. Prostate cancer C42 cells were treated with 10 nM of TGFα-pseudomonas exotoxin chimera (PE) and 500 nM PI3K inhibitor ZSTK474 (ZSTK; FIGS. 6A and 6B) or LY294002 (FIG. 6C) individually and in combination. Caspase activity was measured 3 h and 6 h after treatments began (FIGS. 6A and 6C) and time lapse video-recording of C42 cells treated with 10 nM PE or combination of 10 nM PE and 500 nM ZSTK474 was performed (FIG. 6B). Representative images were taken 2, 5, and 14 h after treatments began. The data demonstrate the unexpected synergistic induction of apoptosis in prostate cancer cells following exposure to the synergistic compositions of the invention.

J591-HSSKLQL-LY294002 pro-drug conjugates will be generated. Conjugation will be performed using the Protein-Protein Crosslinking Kit (Molecular Probes, Eugene, Oreg.) according to the manufacturer's instructions. Briefly, J591 will be incubated with Succinimidyl trans-4-(maleimidylmethyl)-cyclohexane-1-carboxylate (SMCC) in a 1:3 molar ratio and HSSKLQL-LY294002 with Succinimydyl 3-(2-pyridyldithio)propionate (SPDP), in a molar ratio of 1:10 SPDP in 0.1M sodium phosphate, 0.1M NaCl, pH 7.5 buffer for 1.5 hours at room temperature with stirring. We may optionally introduce additional lysine residues (KK) into the HSSKLQ peptide in order to increase the effectiveness of its derivatization while leaving the proteolysis side intact. Derivatized molecules will be purified away from the cross-linking reagents with spin-OUT micro columns (Chemicon). The thiolated derivative of KKHSSKLQL-LY294002 will be incubated in a 5:1 molar ratio (TCEP:HSSKLQL-LY294002) of Tris-(2-carboxyethyl)phosphine, hydrochloride (TCEP) for 15 minutes at room temperature to de-protect the peptide and generate free thiols suitable for conjugation. The J591 and KKHSSKLQL-LY294002 will be combined in a 1:3 molar ratio of for 1 hour at room temperature, followed by 4° C. overnight. Then, N-ethylmalemide (NEM) will be added at a concentration equal to that of the SPDP and incubated for 30 minutes at room temperature, after which the conjugate will be separated from NEM and crosslinkers by gel-filtration chromatography, and stored at −80°.

Efficacy of antibody-fused pro-drug in mice with C42Luc xenografts will be analyzed as described above.

We also will examine constructs where both pro-drugs (PI3K inhibitor and PE) are linked to the same anti-PSMA antibody and test whether anti-tumor efficacy of such conjugates exceeds that of individually conjugated pro-drugs.

Prophetic Treatment of Prostate Cancer in Patients

The following prophetic example is provided to illustrate treatment of androgen-independent metastatic prostate cancer with tumor-targeted, PI3K inhibitor and toxin pro-drugs of the invention. A multi-center, randomized, open-label study can be conducted to evaluate the safety and efficacy of tumor-targeted, PI3K inhibitor and toxin pro-drugs in subjects with androgen-independent metastatic prostate cancer as measured by overall survival compared with best supportive care. In the following example, a Syn2 type inhibitor drug (as exemplified in FIG. 3d) will be tested. A Syn2 inhibitor has an inactive pro-drug of a PI3K inhibitor (such as ZSTK474 or LY294002) attached via a PSA-cleavable peptide linker to an anti-PMSA antibody (such as J591) moiety, which is fused to a toxin (such as PE40).

Subjects are divided into two groups of 150 subjects per treatment group (i) Syn2 inhibitor treatment group (Group I) and (ii) best supportive care (BSC, Group II). Subjects in Group I are administered Syn2 inhibitor for duration of up to 51 weeks. Syn2 inhibitor is administered intravenously over 6 hours once every 3 weeks at 4500 mg/m² for up to 17 doses. One-quarter of the dose is infused over 30 minutes and the remainder over the following five and half hours. Subjects may receive palliative therapies, but not within ±48 hours of a dose of Syn2 inhibitor. In order to assess the efficacy of Syn2 inhibitor treatment, subjects in Group II are not administered any medication that has antitumor effects e.g., chemotherapy or other systemic cytotoxic/cytostatic therapies. However, other appropriate supportive measures and concomitant medications that do not have antitumor effects, such as analgesics, antibiotics, transfusions, hematopoietic colony-stimulating factors (as therapy but not as prophylaxis), erythropoietin, megestrol acetate for appetite stimulation, are administered when appropriate. Subjects in Group I also receive best supportive care.

Tumor assessment is performed at baseline and every 6 weeks for the first 24 weeks and then every 9 weeks until disease progressions are documented. Pharmacokinetic samples are collected from subjects in Group I during cycles 1 and 2. Blood samples for plasma concentrations of Syn2 inhibitor are collected at the following times on day 1 of cycles 1 and 2 from the subjects in Group I at the following time points: predose and immediately before completion of Syn2 inhibitor infusion. Additional pharmacokinetic parameters are measured for a subset of 24 subjects in Group I (area under the curve (AUC), Cₘₐₓ, and T_{1/2} for Syn2 inhibitor). Blood samples are collected from this subset at the following times on day 1 of cycles 1 and 2: predose, 0.5 (immediately before changing the infusion rate), 1, 3, 6 (immediately before completion of Syn2 inhibitor infusion), 6.25, 6.5, 7, 8, 10, 16, 24 hours after the start of Syn2 inhibitor infusion.

The pharmacokinetic parameters for Syn2 inhibitor (day 1 of cycles 1 and 2) are computed for each subject in the 24-subject subset. Efficacy outcomes are evaluated based on the response rate (complete response and partial response), duration of response, progression-free survival, 6- and 12-month survival, and serum PSA levels compared with best supportive care. Syn2 inhibitor treated subjects with androgen-independent metastatic prostate cancer have improved overall survival compared with best supportive care.

### Prophetic Treatment of Breast, Ovarian, Colon and Lung Cancer in Patients

For the treatment of other cancers, the tumor Ag specific antibody moiety in the Syn2 type inhibitor would be specific for an antigen expressed by the tumor of interest. Non-limiting examples of tumor-associated antigens or markers are as follows.

### Breast

### Cancer Antigen 15-3 (CA 15-3)

### MUC-1

epithelial tumor antigen (ETA)

### Ovarian

Cancer antigen 125 (CA-125) is a protein found on the surface of many ovarian cancers.

### Colon

Carcinoembryonic antigen (CEA)
Carbohydrate antigen 19-9 (CA19-9) or sialylated Lewis (a) antigen

### Lung

Carcinoembryonic antigen (CEA)

In addition, for treatment of other cancers, the protease-cleavable linker in Syn2 type inhibitors will be peptide linkers comprising cleavage recognition sites for proteases expressed by particular tumor types of interest or expressed at higher levels in tumor or malignant cells as compared to normal or healthy cells.

Proteases Associated with Cancer

In cancer, altered proteolysis leads to unregulated tumor growth, tissue remodeling, inflammation, tissue invasion, and metastasis. The matrix metalloproteinases (MMPs or matrixins) represent the most prominent family of proteinases associated with tumorigenesis. MMPs are zinc-dependent proteinases and the expression of MMP genes is reported to be activated in inflammatory disorders and malignancy. In addition, there are reports of increased activation and expression of urokinase type plasminogen activator in a variety of disorders such as rheumatoid arthritis, osteoarthritis, atherosclerosis, Crohn's disease, and central nervous system disease, as well as in malignancy. Matrix metalloproteinase-2 (MMP-2) has been found to play an important role in the transformation, migration and invasion of large-cell, undifferentiated lung carcinoma. Examples of proteases expressed at higher levels in malignant as compared to normal cells include MMPs, such as MMP-2, MMP-9, MMP-14, and MT1-MMPs, and UPA.

Matripase is a serine protease known to be associated with breast and colon cancer. Matriptase was initially identified in T-47D human breast cancer cells and is believed to play a role in the metastatic invasiveness of breast cancer. The primary cleavage specificity of matriptase is at arginine and lysine residues, similar to the majority of serine proteases, including trypsin and plasmin. In addition, matriptase, like trypsin, exhibits broad spectrum cleavage activity. Matriptase is co-expressed with its cognate inhibitor, hepatocyte growth factor activator inhibitor 1 (HAI-1; a type 1 integral membrane, Kunitz-type serine protease inhibitor) in many types of normal and malignant tissues of epithelial origin. Several studies suggest that matriptase is over-expressed in a wide variety of malignant tumors including prostate, ovarian, uterine, colon, epithelial-type mesothelioma and cervical cell carcinoma.

### Breast Cancer Associated Proteases

Elevated levels of two related tumor-associated proteases (urokinase-type plasminogen activator (uPA) and its inhibitor, plasminogen activator inhibitor-1 (PAI-1)) are correlated with an increased risk of recurrence after definitive surgical treatment for node-negative breast cancer. Patients who had elevated levels of either protease, had a significantly higher incidence of recurrence without adjuvant chemotherapy.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for synergistically inducing apoptosis in cancer cells in a patient in need thereof comprising administering an effective amount of a PI3K inhibitor and a toxin molecule, in a pharmaceutically acceptable carrier, said PI3K inhibitor and toxin molecule acting synergistically to rapidly induce apoptosis in said cancer cell, said method optionally comprising administration of a chemotherapeutic agent.

2. The method of claim 1, wherein said PI3K inhibitor is selected from the group consisting of LY294002 and biologically active derivatives thereof, LY292223, LY293696, LY293684, LY293646, wortmannin, PX-866, ZSTK474, SF1126, BEZ235, VQD-002, KRX-0401, GSK690693 and XL147 and prodrugs thereof.

3. The method of claim 1, wherein said toxin is selected from the group consisting of Pseudomonas exotoxin (PE) A, PE40, ricin, ricin A-chain, diphtheria toxin, abrin, abrin A chain, modeccin A chain, alpha-sarcin, gelonin, mitogellin, retstrictocin, phenomycin, enomycin, curicin, crotin, and calicheamicin and prodrugs thereof.

4. A method as claimed in claim 1 to claim 3, wherein said cancer is selected from the group consisting of prostate cancer, ovarian cancer, breast cancer, lung cancer, brain cancer, liver cancer, stomach cancer, pancreatic cancer, and esophageal cancer.

5. The method of claim 1, wherein said cancer is prostate cancer, said PI3K inhibitor is a prodrug of LY294002 or ZSTK474 comprising a PSA cleavable linker and said toxin is Pseudomonas exotoxin (PE) A or PE40 operably linked to an antibody thereby forming an immunotoxin which has binding specificity for an antigen present on a prostate cancer cell, said immunotoxin optionally comprising a PSA cleavable linker.

6. The method of claim 5, wherein said antigen is selected from the group consisting of PMSA, PCA, MUC1, Epidermal growth factor receptor, platelet-derived growth factor, platelet-derived growth factor receptor, urokinase plasminogen activator, and urokinase plasminogen activator receptor.

7. The method of claim 5, wherein said PSA cleavable linker is selected from the group consisting of HSSKLQL (SEQ ID NO: 1), CHSSKLQG (SEQ ID NO: 2), EHSSKLQ (SEQ ID NO: 3), QNKISYQ (SEQ ID NO: 4), INKISYQ (SEQ ID NO: 5) and ATKSKQH (SEQ ID NO: 6 (SEQ ID NO: 6).

8. The method of claim 5, wherein each of said inhibitor and said toxin are operably linked to said antibody thereby enhancing prostate cancer cell targeting.

9. The method of claim 1, wherein said inhibitor and said toxin are administered simultaneously.

10. The method of claim 1, wherein said inhibitor and said toxin are administered sequentially.

11. The method of claim 1, comprising administration of at least one chemotherapeutic agent selected from the group consisting of placitaxel (Taxol®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

12. The method of claim 1, wherein said inhibitor and said toxin are administered via a route selected from the group consisting of systemic administration, parenteral administration, direct injection at a tumor site, intraperitoneal administration.

13. The method of claim 1, wherein said inhibitor and said toxin are effective at sub-therapeutic doses.

14. A synergistic anti-prostate cancer formulation comprising:
i) a LY294002 prodrug or ZSTK474 prodrug operably linked to a PSA cleavable linker which is effective to inhibit PI3K activity;
ii) a Pseudomonas exotoxin (PE) A or PE40 operably linked to an antibody which has binding specificity for PMSA antigen thereby forming an immunotoxin, said immunotoxin optionally comprising a PSA cleavable linker, each of i) and
ii) being present in a pharmaceutically acceptable carrier.

15. A synergistic anti-prostate cancer formulation comprising:
i) ZSTK474 operably linked to a PSA cleavable linker and
ii) Pseudomonas exotoxin (PE) A operably linked to J591, each of i) and ii) being present in a pharmaceutically acceptable carrier.

16. A synergistic anti-prostate cancer formulation comprising:
i) LY294002 operably linked to a PSA cleavable linker and
ii) Pseudomonas exotoxin (PE) A operably linked to J591, each of i) and ii) being present in a pharmaceutically acceptable carrier.

17. The formulation as claimed in claim 15 or claim 16 wherein said ZSTK474 or LY294002 and said toxin are operably linked by an antibody having immunospecificity for an antigen present on prostate cancer cells.


SEQUENCE LISTING (7 sequences)

SEQ ID NO: 1 - protein, 7 aa, Artificial Sequence
  Linker
HSSKLQL

SEQ ID NO: 2 - protein, 8 aa, Artificial Sequence
  Linker
CHSSKLQG

SEQ ID NO: 3 - protein, 7 aa, Artificial Sequence
  Linker
EHSSKLQ

SEQ ID NO: 4 - protein, 7 aa, Artificial Sequence
  Linker
QNKISYQ

SEQ ID NO: 5 - protein, 7 aa, Artificial Sequence
  Linker
INKISYQ

SEQ ID NO: 6 - protein, 7 aa, Artificial Sequence
  Linker
ATKSKQH

SEQ ID NO: 7 - protein, 9 aa, Artificial Sequence
  Linker
KKHSSKLQL